(19) United States
(12) Patent Application Publication
(10) Pub. No.: US 2025/0387424 A1
(43) Pub. Date: Dec. 25, 2025

(54) PHARMACEUTICAL COMPOSITION COMPRISING A3 ADENOSINE RECEPTOR AGONIST FOR TREATMENT OF PSORIASIS

(71) Applicant: CAN-FITE BIOPHARMA LTD., Petah Tikva (IL)

(72) Inventor: Pnina FISHMAN, Herzliya (IL)

(21) Appl. No.: 18/879,758

(22) PCT Filed: Jun. 29, 2023
(86) PCT No.: PCT/IL2023/050669

§ 371 Date: Dec. 28, 2024

(30) Foreign Application Priority Data
Jun. 29, 2022 (IL) 294409

(51) Int. Cl. A61K 31/7076; A61P 17/06

(52) U.S. Cl. CPC A61K 31/7076; A61P 17/06

(57) ABSTRACT

The invention concerns methods and pharmaceutical compositions for use in the treatment of psoriasis comprising as an active ingredient piclidenoson (IB-MECA) in an amount suitable for a daily dose administration of about 6 milligrams (mg).

### FIELD OF THE INVENTION

This invention relates to the treatment of psoriasis, particularly plaque psoriasis, with piclidenoson.

### BACKGROUND OF THE INVENTION

Psoriasis is a common, chronic condition causing skin inflammation which impacts quality of life for patients which often experience flares and remissions throughout their lifetime. Psoriasis is an autoimmune inflammatory disease in which cutaneous plaques are formed due to the resistance of skin keratinocytes to apoptosis. Cytokines including TNF-α, IL-17, and IL-23 induce the continuous proliferation of keratinocytes, thereby playing a key role in disease pathogenesis [Zhou et al. Cell Death Dis 13, 81 (2022)]. Although during the last 2 decades, biological therapies targeting these inflammatory cytokines have been successfully implemented as psoriasis treatment, their toxicity/side effects, loss of efficacy over time, disease recurrence after their discontinuation, and cost prompted the exploration of novel strategies for psoriasis treatment [Al-Janabi, A. & Yiu, Z. Z. N. Psoriasis (Auckl) 12, 1-14 (2022)].

A3 adenosine receptor (A3AR) is overexpressed in the skin and peripheral blood mononuclear cells of psoriasis patients.

WO 2011/027348 discloses pharmaceutical compositions comprising the A₃AR agonist CF101 (methyl 1-[N⁶-(3-iodobenzyl)-adenin-9-yl]-β-D-ribofuronamide, IB-MECA,) for use in the treatment of psoriasis in a daily dose of 4 milligrams (mg).

### SUMMARY OF THE INVENTION

In a first of its aspects, the present invention provides a pharmaceutical composition for use in the treatment of psoriasis comprising as an active ingredient 1-[N⁶-(3-iodobenzyl)-adenin-9-yl]-β-D-ribofuronamide (IB-MECA) in an amount suitable for a daily dose administration of about 6 milligrams (mg).

In one embodiment, the pharmaceutical composition for use is in a dosage form suitable for oral administration.

In one embodiment, the pharmaceutical composition for use is in a dosage form suitable for administration once or twice a day, the total daily dose being about 6 mg.

In one embodiment, the pharmaceutical composition for use is in an oral solid dosage form.

In one embodiment, the solid dosage form comprises 3 mg IB-MECA which is administered twice a day.

In one embodiment, the dosage form is selected from pills, tablets, and capsules.

In another aspect, the present invention provides IB-MECA for use in treatment of psoriasis in an amount suitable for providing a subject in need of the treatment with a total daily dose of IB-MECA being about 6 mg.

In one embodiment, the daily dose is formulated for administration once or twice a day.

In one embodiment, the daily dose is formulated in a form suitable for oral administration.

In one embodiment, the IB-MECA for use is formulated in a solid dosage administration form.

In one embodiment, the solid dosage form is selected from the group consisting of pills, tablets, and capsules.

In one embodiment, the dosage form is suitable for administration of 3 mg IB-MECA, which is administered twice a day.

In another aspect, the present invention provides a package comprising the pharmaceutical composition of the invention as defined above and instructions for administration of the pharmaceutical composition to a subject in need of treatment of psoriasis at a daily dose of IB-MECA of about 6 mg.

In one embodiment, the pharmaceutical composition comprises 3 mg IB-MECA and said instructions comprises administration of the pharmaceutical composition to the subject twice a day.

In another aspect, the present invention provides a method comprising administering to a subject in need of psoriasis treatment a daily dose of about 6 mg of IB-MECA, wherein the subject is being treatment for psoriasis.

In one embodiment, said subject is administered with IB-MECA once or twice a day.

In one embodiment, the IB-MECA is orally administered to said subject.

In one embodiment, the IB-MECA is administered in a form selected from the group consisting of pills, tablets, and capsules.

In one embodiment, the method comprises administering IB-MECA to the subject twice daily, each administration dose comprising 3 mg IB-MECA.

In one embodiment, the psoriasis is moderate to severe plaque psoriasis.

In another aspect, the present invention provides a method for treatment of psoriasis comprising administering 1-[N⁶-(3-iodobenzyl)-adenin-9-yl]-β-D-ribofuronamide (IB-MECA) or a pharmaceutical composition comprising IB-MECA to a patient in need thereof in a daily dose of about 6 milligrams (mg).

In one embodiment, said IB-MECA or said pharmaceutical composition are administered orally.

In one embodiment, said IB-MECA or said pharmaceutical composition are administered once or twice a day, the total daily dose being about 6 mg.

In one embodiment, said IB-MECA or said pharmaceutical composition are administered as a solid dosage form comprising 3 mg IB-MECA which is administered twice a day.

In one embodiment, the solid dosage form is selected from pills, tablets, and capsules.

In one embodiment, said psoriasis is moderate to severe plaque psoriasis.

### BRIEF DESCRIPTION OF THE DRAWINGS

To better understand the subject matter that is disclosed herein and to exemplify how it may be carried out in practice, embodiments will now be described, by way of non-limiting examples only, with reference to the accompanying drawings, in which:

FIG. 1 is a schematic representation of the clinical study design.

FIG. 2 is a graph showing the proportion of patients achieving PASI 75 by treatment arm over time (weeks).

FIG. 3 is a graph showing the proportion of patients achieving PGA2 by treatment arm over time (weeks).

FIG. 4 is a graph showing the proportions of patients with improvements from baseline (BL) in psoriasis disability index (PDI) at Week 32 by study arm (per protocol (PP) population).

FIG. 5A-B is a graph showing Cₘₐₓ (A) and AUC (B) for piclidenoson 2 mg and 3 mg BID over time (weeks). Error bars represent SE.

### DETAILED DESCRIPTION OF EMBODIMENTS

The present invention is based on results of a phase 3 clinical trial in patients with moderate to severe plaque psoriasis. The efficacy of oral administration of piclidenoson (also referred to herein as CF-101 or IB-MECA) in ameliorating disease symptoms in psoriasis patients was tested.

The randomized, placebo- and active-controlled, double-blind phase 3 trial (also referred to herein as the COMFORT-1 trial) randomized patients (3:3:3:2) to piclidenoson 2 mg twice a day (referred to herein as BID), piclidenoson 3 mg BID, apremilast 30 mg BID, or placebo. At Week 16, patients in the placebo arm were re-randomized (1:1:1) to piclidenoson 2 mg BID, piclidenoson 3 mg BID, or apremilast 30 mg BID. The primary endpoint was the proportion of patients achieving ≥75% improvement in Psoriasis Area and Severity Index (PASI) from baseline (PASI-75) at Week 16 vs. placebo.

A total of 529 patients were randomized and received ≥1 dose of piclidenoson (safety population). The efficacy analysis population for the primary endpoint included 426 patients (piclidenoson 2 mg BID, 127; piclidenoson 3 mg BID 103; apremilast, 118; placebo, 78). The study results demonstrated a marked effect of the 3 mg dose of IB-MECA, administered orally twice daily (namely a total daily dose of 6 mg) in ameliorating disease manifestation in these patients.

Namely, the primary endpoint was met with the 3 mg BID dose: PASI 75 rate of 9.7% vs. 2.6% for piclidenoson vs. placebo, p=0.037. The PASI responses with piclidenoson continued to increase throughout the study period in a linear manner. At week 32, analysis in the per protocol population showed that a greater proportion of patients in the piclidenoson 3 mg BID arm (51/88, 58.0%) achieved improvement from baseline in psoriasis disability index (PDI) compared to apremilast (59/108, 55.1%), and the test for noninferiority trended towards significance (p=0.072). The safety profile of the 3 mg dose of piclidenoson was excellent and better than apremilast. Since psoriasis is a chronic disease which requires in most cases lifelong treatment, the favorable safety profile is of utmost importance. The clinically meaningful improvements demonstrated in the phase 3 trial described herein, in both skin symptoms and quality of life, underline the highly favorable therapeutic index of the 3 mg Piclidenoson dose in psoriasis.

Piclidenoson is an A3 adenosine receptor agonist (A3AR) small molecule. Without wishing to be bound by theory, Piclidenoson's mechanism of action entails inhibition of the inflammatory cytokines, interleukin 17 and 23 (IL-17 and IL-23) and the induction of apoptosis of patients' skin cell keratinocytes involved with the disease pathogenicity.

In the context of the present disclosure the term “psoriasis” encompasses any form of psoriasis including, without being limited thereto, plaque psoriasis; pustular psoriasis (including arthritic psoriasis or psoriatic arthritis); Guttate psoriasis; inverse or flexural psoriasis; erythroderma psoriasis. Further, in the context of the present invention, when referring to “psoriasis” it is meant to include any degree of psoriasis, including, mild, moderate, and severe psoriasis. In a specific embodiment the term “psoriasis” refers to moderate to severe plaque psoriasis. In a specific embodiment the term “psoriasis” refers to moderate to severe chronic plaque psoriasis.

As indicated above, there are several forms of psoriasis, and each form has unique characteristics that allow dermatologists to visually identify psoriasis to determine what type, or types, of psoriasis is present. Sometimes a skin biopsy will be performed to confirm the diagnosis. The main types of psoriasis include the following:

Plaque Psoriasis (reddened areas a few inches across covered by silvery scales)
Pustular Psoriasis (blisters of noninfectious pus on red skin)—Arthritic Psoriasis or Psoriatic Arthritis
Guttate Psoriasis (small, red spots on the skin)
Inverse or Flexural Psoriasis (shiny, red patches in areas of friction such as in the folds of skin in the groin, the armpits or under the breasts)
Erythroderma Psoriasis (reddening and scaling of most of the skin).

In the context of the present disclosure the term “treatment” includes any improvement in one or more objective parameters that are used to assess a psoriatic state (severity) in clinical trials, namely redness, thickness and scaliness of psoriatic lesions. Based on these parameters, several tools for assessing the effectiveness of treatment of psoriasis have been developed. The assessment tools include traditional assessment tools such as the Psoriasis Area and Severity Index (PASI), the Physician Global Assessment (PGA), as well as more recent assessment tools, such as the National Psoriasis Foundation Psoriasis Score (NPF-PS), the Physical Static Global Assessment (PSGA) and Overall Lesion Assessment (OLA) [S. R. Feldman and G. G. Krueger Psoriasis assessment tools in clinical trials, Ann Rheum. Dis. 64 (Suppl. II):ii65-ii68 (2005)].

PASI and PGA are the two most used tools in assessing psoriasis activity and in following clinical response to treatment. The PASI assessment tool evaluates the degree of erythema, thickness and scaling of psoriatic plaques and estimates the extent of involvement of each of these components in four separate body areas (head, trunk, upper and lower extremities). The PASI composite score ranges from 0-72. The PGA assessment tool is a six-point score that summarizes the overall erythema, scaling, and thickness and the extend of plaques relative to a baseline assessment, the scores including worse, poor (0-24%), fair (25-49%), good (50-74%), excellent (75-99%) and cleared (100%). [Alice B Gottlieb et al. The National Psoriasis Foundation Psoriasis Score System versus the Psoriasis Area Severity Index and Physician's Global Assessment: a comparison, Journal of Drugs in Dermatology June 2003].

In a specific embodiment, the invention pertains to the treatment of a psoriasis patient with a body surface area (BSA) involvement ≥10%, optionally having a PASI score ≥12, a static PGA ≥3, and having psoriasis for ≥6 months.

The present invention provides a pharmaceutical composition comprising as active ingredient IB-MECA for use in treating psorisasis, IB-MECA for use in treatment and a method for treatment of psoriasis, the composition, IB-MECA for use and method being characterized in that the active ingredient, i.e., IB-MECA is administered to achieve a total daily dose of about 6 mg.

The term “daily dose” should be understood to encompass the amount of the active ingredient, namely, IB-MECA being administered per day to the subject in need thereof. The daily dose may encompass a single daily administration or more than one administration per day, provided that the total amount of IB-MECA received by the subject per day is about 6 mg.

In line with the above, the pharmaceutical composition may be formulated for a single daily administration, in which case the amount of IB-MECA in the composition is about 6 mg, or for administration twice a day, in which case the amount of IB-MECA is about 3 mg. Similarly, the composition may be formulated for 3- or 4-times daily administration in which case the dosage form will comprise, respectively, about 2 and 1.5 mg of IB-MECA.

In a preferred embodiment, the active ingredient, namely, IB-MECA, is formulated in a form suitable for oral administration. However, in some embodiments the composition may be formulated for nasal administration, may be in the form of an inhaled formulation, may in the form of a suppository or may even be formulated for parenteral administration.

Oral administration, in the context of the present invention, includes any one of (a) liquid solutions, such as an effective amount of IB-MECA dissolved in diluents, such as water, saline or even orange juice, (b) solid and semi solid forms, (c) powders; (d) suspensions in an appropriate liquid; and (e) suitable emulsions. IB-MECA will typically be formulated in a dosage form suitable to achieve the desired daily dose of 6 mg.

Liquid forms may include diluents, such as water and alcohols, for example, ethanol, benzyl alcohol, and polyethylene alcohols, either with or without the addition of a pharmaceutically acceptable surfactant, suspending agent, or emulsifying agent.

Solid forms, which are a preferred administration form in the context of the invention, may include, without being limited thereto, pills, tablets, including immediate and modified (controlled) release tablets, uncoated and coated tablets (including enteric coating), chewable tablets, bi or multi layer tablets; pellets; capsules, including soft gelatin gel capsules and hard-shelled gelatin capsules; powders including granules and oral powders for reconstitution, lozenges, cachets.

The capsules may include, for example, surfactants, lubricants, and inert fillers, such as lactose, sucrose, calcium phosphate, and corn starch. The tablets may include one or more of lactose, sucrose, mannitol, corn starch, potato starch, alginic acid, microcrystalline cellulose, acacia, gelatin, guar gum, colloidal silicon dioxide, croscarmellose sodium talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. The lozenges may comprise IB-MECA in a flavor, such as sucrose and acacia or tragacanth, as well as pastilles comprising IB-MECA in an inert base, such as gelatin and glycerin, or sucrose and acacia, emulsions, gels, and the like containing, in addition to IB-MECA, such carriers as are known in the art.

The pharmaceutical composition further comprises at least one pharmaceutically acceptable excipient to facilitate oral delivery of IB-MECA.

In one embodiment, the pharmaceutical composition is in the form of a tablet. A tablet may be made by compression or molding, optionally with one or more of said excipients. Compressed tablets may be prepared by compressing in a suitable machine IB-MECA in a free-flowing form, e.g., a powder or granules, optionally mixed with the excipient(s), e.g., binders, lubricants, inert diluents, surface active or dispersing agents. Molded tablets may be made by molding in a suitable machine, a mixture of the powdered IB-MECA with any suitable carrier.

A person versed in the art of pharmacy and formulation technologies of pharmaceutical ingredients will be able to devise a myriad of different formulations for oral or other forms of administration.

In accordance with the present disclosure, IB-MECA may be administered once, twice or several times a day. In one embodiment, IB-MECA is administered once a day, the dosage form including about 6 mg of IB-MECA. In another embodiment, IB-MECA is administered twice a day, each administration dosage form including about 3 mg of IB-MECA (to achieve the total daily amount of 6 mg).

The present disclosure also provides the use of IB-MECA in the treatment or in the preparation of a pharmaceutical composition for the treatment of psoriasis, the administration dose being formulated in a form suitable for a daily administration of about 6 mg of IB-MECA.

Finally, the present disclosure provides IB-MECA for use in the treatment of psoriasis, said IB-MECA being administered to a subject having psoriasis in a daily dose amount of about 6 mg.

The term “about” should be understood to encompass a dose that is within a range of 10% less or 10% more (“±10%”) than the indicated dose.

### DESCRIPTION OF NON-LIMITING EXAMPLES

### Materials and Methods

### Study Design

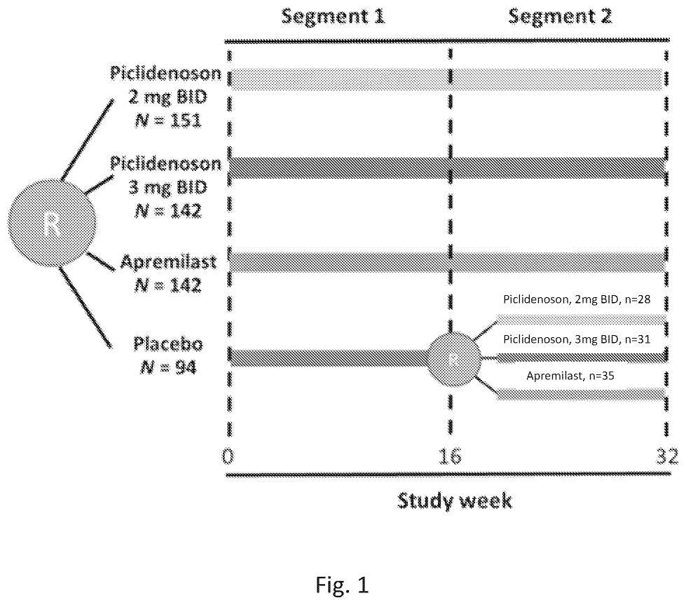

The COMFORT-1 study was a phase 3 multicenter (with sites in Bosnia, Bulgaria, Canada, Israel, Moldova, Poland, Romania, and Serbia) randomized, double-blind, controlled clinical trial in moderate-to-severe plaque psoriasis (ClinicalTrials.gov identifier: NCT03168256). The trial had 2 segments (FIG. 1). The first segment (Weeks 0-16) included 4 treatment arms: two arms with piclidenoson (2 mg or 3 mg BID), one with the active comparator apremilast (dose-titrated over 6 days to 30 mg BID, according to the label), and a placebo arm. Eligible patients were randomly assigned to these arms in a 3:3:3:2 ratio. Blinding was maintained using a double-dummy technique. The second segment (Weeks 16-32) included 3 treatment arms, as at Week 16, the patients in the placebo arm were re-randomized to piclidenoson 2 mg BID, piclidenoson 3 mg BID, or apremilast (dose-titrated over 6 days to 30 mg BID) in a 1:1:1 ratio and treated through Week 32. Those originally assigned to piclidenoson or apremilast remained on their initially assigned treatment through Week 32. Blinding was maintained through Week 32. Patients were evaluated every 2 weeks for efficacy and safety. The primary efficacy endpoint was assessed at Week 16, and the secondary endpoints were assessed at Weeks 16 and 32. Pharmacokinetic (PK) sampling was performed in Weeks 0, 8, and 16.

The study was approved by all relevant national regulatory authorities and local Ethics Committees/Institutional Review Boards. The study was conducted in accordance with the Declaration of Helsinki and written informed consent was obtained from all patients.

### Study Patients

The study population included male and female patients aged 18-80 years with a diagnosis of moderate-to-severe chronic plaque psoriasis with a body surface area (BSA) involvement ≥10% who were candidates for systemic treatment or phototherapy. Main inclusion criteria were having a PASI score ≥12, a static PGA ≥3, and having psoriasis for 26 months. Key exclusion criteria included prior treatment with apremilast within 4 weeks of the baseline (BL) visit, or contraindication to apremilast, certain prior/concomitant treatment such as systemic retinoids, corticosteroids, tofacitinib, or immunosuppressive agents within 4 weeks of the BL visit; an approved/investigational biological agent within a period of time that was either equal to 5 times its circulating half-life, or 30 days prior to the BL visit (whichever was longer); high potency dermatological corticosteroids (Class I-III in the United States, Class III-IV in Europe), vitamin D analogs, keratolytics, or coal tar (other than on the scalp, palms, groin, and/or soles) within 2 weeks of the BL visit; ultraviolet/Dead Sea therapy within 4 weeks of the BL visit, or anticipated need for these therapies during the study period. Renal or hepatic dysfunction, uncontrolled concomitant illness, and pregnancy/lactation.

### Efficacy and Safety Assessment

The primary endpoints included the proportion of patients achieving PASI 75 in the piclidenoson 2 mg and 3 mg BID arms vs. placebo at Week 16 (superiority) and safety. Secondary endpoints at Week 16 compared the piclidenoson arms to placebo and included the proportion of patients achieving PASI 50, the proportion of patients achieving a score of 0 or 1 in PGA2 (a PASI-based measure calculated using the intensity grading of components of the PASI score; specifically, it is the average of the PASI erythema, infiltration, and desquamation scores) [Gordon, K. B. et al. N Engl J Med 373, 136-144 (2015)], and the proportion of patients experiencing an improvement from BL in Psoriasis Disability Index (PDI). Additional secondary endpoints were evaluated at Week 32 and compared the piclidenoson arms to apremilast. These included the proportion of patients achieving PASI 75, PASI 50, PGA2 of 0 or 1, and the proportion of patients experiencing an improvement from BL in PDI. PK analysis, including calculating the peak concentration of the drug in the bloodstream after administration (Cₘₐₓ), was conducted for Weeks 0, 8, and 16.

Safety assessments included treatment emergent adverse events (TEAEs) and changes in vital signs, physical examination, clinical laboratory tests (liver, kidney, hematology, chemistry, and urinalysis), and electrocardiogramdings.

### Statistical Analysis

Overall, 4 analysis populations were defined: The safety population included all patients who received ≥1 dose of piclidenoson; the intent-to-treat (ITT) population included all those in the safety population with at least one PASI score recorded post-BL and excluded patients who withdrew prior to Week 16 due to COVID-19-related study suspension; the modified ITT (mITT) population, which was used for the Week 16 efficacy analyses, included all ITT patients except one patient in the placebo arm who was excluded due to major protocol violations that were not captured in the original data; and lastly, the per protocol (PP) population, which was used for the Week 32 analyses, and included all ITT patients who had no major protocol violations on or before Week 16 and who completed Week 16 of the study. Exclusion from the PP population was finalized prior to any unblinded analyses. The analyses conducted for Week 32 were performed separately for patients who were initially randomized to piclidenoson or apremilast and for those who were initially randomized to the placebo group.

Descriptive statistics were used to summarize patient characteristics and safety. TEAEs were reported by treatment group for each System Organ Class (SOC) and Preferred Term (PT), as defined in the Medical Dictionary for Regulatory Activities (MedDRA), version 23.0. If a patient had more than one TEAE with the same PT, the patient was counted once.

For the efficacy analyses, missing values were imputed. For the primary efficacy analysis missing values due to discontinuation were considered a non-response post-discontinuation and were referred to as Non-Responder Imputation (NRI); intermediate missing values were imputed using Last Observation Carried Forward (LOCF). Missing values for the secondary efficacy analyses were also imputed using NRI. p values for the primary efficacy analysis and for the secondary analyses comparing piclidenoson to placebo were determined with the normal approximation for comparing 2 binomial proportions (NAB). Secondary analyses comparing piclidenoson to apremilast were noninferiority analyses with a noninferiority margin of 10% using NAB. Data are presented as proportions/means±SE.

All statistical tests were 2-sided and p<0.05 was considered statistically significant.

### Results

A Phase III, multicenter, randomized, placebo- and active-controlled, double-blind study was conducted to assess the efficacy and safety of Piclidenoson (IB-MECA, CF101) in patients with moderate to severe plaque psoriasis.

### Patients

A total of 701 patients with moderate-to-severe plaque psoriasis were screened. Patients who met the eligibility criteria (N=529) were randomized to piclidenoson 2 mg BID (N=151), piclidenoson 3 mg BID (N=142), apremilast (N=142), or placebo (N=94).

Overall, nearly a third of all patients (n=158, 29.9%) across all treatment arms discontinued treatment in the first segment of the study (Weeks 0-16), most notably due to the COVID-19-related study suspension (n=93 across all arms, 17.6%). Additional reasons for study discontinuation included patients' requests (n=39, 7.4%) and TEAEs (n=7, 1.3%).

The baseline patient and disease characteristics of the study safety population by treatment are presented in Table 1, demonstrating balanced distribution across the study arms. Most of the study patients were males, the median age (range) for the entire study population was 49 (19-81) years, and all but 1 were White. The median (range) duration of disease for all patients was 12 (0-67) years and was similar between the treatment arms.

TABLE 1
Baseline patient and disease characteristics

| | Piclidenoson | | Piclidenoson | | | | | |
|---|---|---|---|---|---|---|---|---|
| | 2 mg BID | | 3 mg BID | | Apremilast | | Placebo | |
| Parameter | N = 151 | | N = 142 | | N = 142 | | N = 94 | |
| Male, n (%) | 85 | (56.3%) | 94 | (66.2%) | 95 | (66.9%) | 55 | (58.5%) |
| Age in years, | 50 | (20-78) | 49 | (19-74) | 47.5 | (19-81) | 47 | (19-77) |
| median (range) | | | | | | | | |
| Weight in kg, | 82 | (45-135) | 82 | (48-141) | 83 | (53-136) | 82 | (41-138) |
| median (range) | | | | | | | | |
| BMI kg/m², | 28.3 | (19.0-51.0) | 27.9 | (16.5-41.9) | 27.6 | (18.4-42.1) | 27.5 | (16.2-43.1) |
| median (range) | | | | | | | | |
| Race, n (%): | | | | | | | | |
| White/Caucasian | 150 | (99.3%) | 142 | (100.0%) | 142 | (100.0%) | 94 | (100.0%) |
| Other | 1 | (0.7%) | 0 | (0.0) | 0 | (0.0) | 0 | (0.0) |
| Ethnicity n (%) | | | | | | | | |
| Hispanic/Latino | 1 | (0.7%) | 1 | (0.7%) | 2 | (1.4%) | 0 | (0.0%) |
| Not | 150 | (99.3%) | 141 | (99.3%) | 140 | (98.6%) | 94 | (100.0%) |
| Hispanic/Latino | | | | | | | | |
| Duration of | 12 | (0-61) | 10 | (1-47) | 13 | (0-67) | 11.5 | (1-45) |

disease in years,
median (range)

BID, twice a day;
BMI, body mass index

Efficacy Vs. Placebo (Week 16)

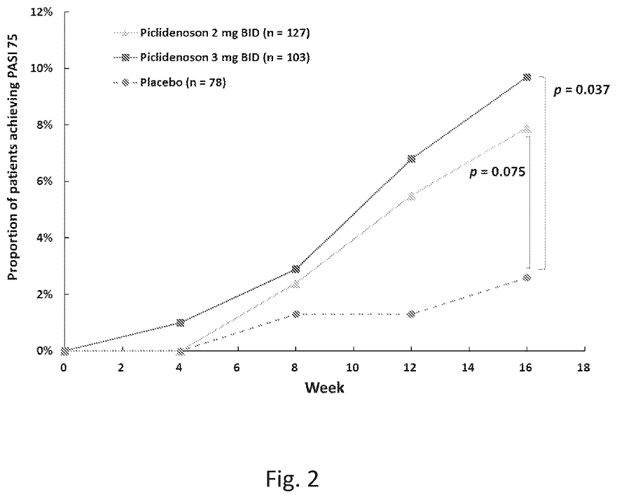

The primary endpoint of the study was met. At week 16, patients receiving Piclidenoson 3 mg demonstrated statistically significant improvement when compared with placebo in the mITT population, as measured by the Psoriasis Area and Severity Index (PASI) 75 response: Piclidenoson 3 mg: 9.7%±1.8% vs. placebo: 2.6%±1.8% (p=0.037) (FIG. 2). Although a greater proportion of patients in the piclidenoson 2 mg BID arm achieved PASI 75 compared to placebo at Week 16 in the mITT population, the difference was not statistically significant (7.9%±2.4% vs. 2.6%±1.8%, p=0.075). For both dosages, the proportion of patients improved linearly over time (FIG. 2).

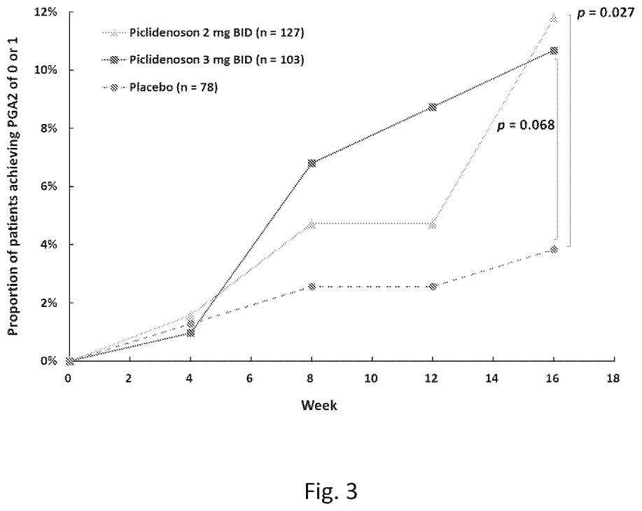

For the secondary endpoint of the proportion of patients achieving PGA2 at Week 16 in the mITT population (FIG. 3), the proportion was greater with piclidenoson 3 mg BID (10.7%±3.0%) and 2 mg BID (11.8%±2.8%) than with placebo (3.8%±2.2%). The observed difference between piclidenoson 3 mg BID and placebo trended towards statistical significance (p=0.068) and was significant for the 2 mg BID dose (p=0.027).

Other secondary endpoints evaluated at Week 16 on the mITT population, including the proportion of patients achieving PASI 50 and the proportion of patients with improvement from BL in PDI did not demonstrate statistically significant superiority of either dose of piclidenoson over placebo. The proportion of patients achieving PASI 50 was 19.4% with piclidenoson 3 mg BID vs. 15.4% for placebo (p=0.475). It was 26.0% for piclidenoson 2 mg BID, and the difference vs. placebo trended towards statistical significance (p=0.060). The proportions of patients achieving PDI improvement from BL was 58.3%, 63.3% and 60.3% for the 3 mg BID, 2 mg BID, and placebo, respectively (p=1.00 and p=0.70 for the respective comparisons vs. placebo).

Efficacy Vs Apremilast in Patients Who were Initially Randomized to Active Treatment (Week 32)

Evaluating noninferiority of piclidenoson vs. apremilast was performed at Week 32 on the PP population and included the patients who were initially randomized to active treatment. For the proportions of patients achieving PASI 75, noninferiority was not established for 3 mg BID vs. apremilast (17.0% vs. 26.2% respectively, p=0.881), or established piclidenoson 2 mg BID vs. apremilast (20.8% vs. 26.2% respectively, p=0.429). The proportions of patients achieving PASI 50 were lower with both piclidenoson doses compared to apremilast, and noninferiority was not established (19.3%, 17.0%, and 25.2% for piclidenoson 3 mg BID, 2 mg BID, and apremilast, respectively; p=0.49 and p=0.75 for the respective comparisons vs. apremilast). Similarly, the proportions of patients with PGA2 of 0 or 1 were also lower (both doses) compared to apremilast, and noninferiority was not established (15.9%, 18.9%, and 24.3% for piclidenoson 3 mg BID, 2 mg BID, and apremilast, respectively; p=0.777 and p=0.417 for the comparisons vs. apremilast, respectively).

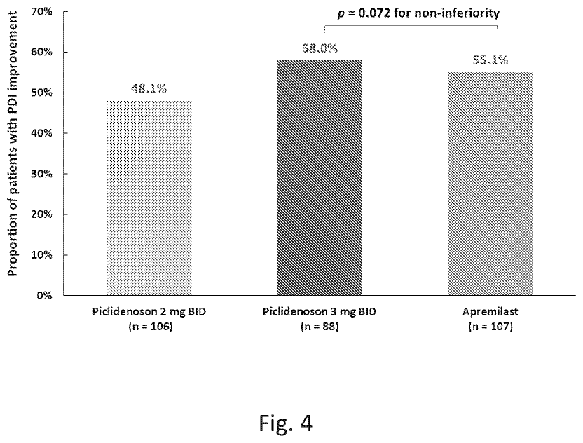

A greater proportion of patients in the piclidenoson 3 mg BID arm achieved improvement from BL in PDI compared to apremilast (58.0% vs. 55.1%). The test for noninferiority trended towards significance (p=0.072, FIG. 4). The corresponding proportion in the 2 mg BID was 48.1%, and noninferiority vs. apremilast was not established (p=0.66).

### Safety

Piclidenoson 2 mg BID and 3 mg BID were well-tolerated throughout the 32-week study period, with no increased TEAE frequency in the higher piclidenoson dose (Table 2). The safety profile of both piclidenoson dosages was generally comparable to that of placebo, which was used only from Week 0 through Week 16. Furthermore, the safety profile of piclidenoson was more favorable than that of apremilast, which was associated with a higher frequency of nervous system disorders (9.6% for apremilast vs. 1.7% for each piclidenoson dose) and gastrointestinal disorders (7.3% for apremilast vs. 2.8% for piclidenoson 2 mg BID and 1.2% for piclidenoson 3 mg BID). The only TEAEs occurring in >2% of patients in the piclidenoson arms were nasopharyngitis (2.2% and 2.9% in the 2 mg BID and 3 mg BID arms, respectively) and urinary tract infection (3.4% in the 2 mg BID arm) (Table 2). Also, no cumulative risk with piclidenoson was observed upon treatment beyond Week 16, when all patients received active treatment.

Overall, there were 3 serious TEAE in patients receiving active treatment including 1 in a patient receiving piclidenoson 2 mg BID (arterial embolism), 1 in a patient receiving piclidenoson 3 mg BID (osteomyelitis), and 1 in a patient receiving apremilast (pneumonia); none were considered by the investigator to be drug related. Nine patients withdrew from the study due to 10 TEAEs (7 in Weeks 0-16 and 2 in Weeks 16-32), including 1 each in the placebo and apremilast arms, 2 in the piclidenoson 2 mg BID arm, and 5 in the piclidenoson 3 mg BID arm. None of the events were considered by the investigator to be drug related. Electrocardiogra showed no clinically significant drug effects on QTcF or other parameters during treatment across all arms. No other safety events of note occurred, and no deaths were reported throughout the study period.

TABLE 2
Incidence of TEAE during Weeks 0-32 in the safety population ^{a}

| MedDRA System | Piclidenoson | | Piclidenoson | | | | | |
|---|---|---|---|---|---|---|---|---|
| Organ Class and | 2 mg BID | | 3 mg BID | | Apremilast | | Placebo | |
| Preferred Term n (%) | N = 179^{b} | | N = 173^{c} | | N = 177^{d} | | N = 94^{e} | |
| Any | 50 | (27.9%) | 41 | (23.7%) | 55 | (31.1%) | 32 | (34.0%) |
| Cardiac disorders | 3 | (1.7%) | 2 | (1.2%) | 2 | (1.1%) | 2 | (2.1%) |
| GI disorders: | | | | | | | | |
| Any | 5 | (2.8%) | 2 | (1.2%) | 13 | (7.3%) | 1 | (1.1%) |
| Diarrhea | 1 | (0.6%) | 1 | (0.6%) | 5 | (2.8%) | 0 | (0.0%) |
| Nausea | 1 | (0.6%) | 0 | (0.0%) | 4 | (2.3%) | 1 | (1.1%) |
| General disorders and | 4 | (2.2%) | 5 | (2.9%) | 4 | (2.3%) | 4 | (4.3%) |
| administration site | | | | | | | | |
| conditions | | | | | | | | |
| Infections/infestations: | | | | | | | | |
| Any | 20 | (11.2%) | 14 | (8.1%) | 19 | (10.7%) | 15 | (16.0%) |
| Nasopharyngitis | 4 | (2.2%) | 5 | (2.9%) | 2 | (1.1%) | 7 | (7.4%) |
| Urinary tract infection | 6 | (3.4%) | 0 | (0.0%) | 3 | (1.7%) | 1 | (1.1%) |
| Investigations: | | | | | | | | |
| Any | 5 | (2.8%) | 10 | (5.8%) | 13 | (7.3%) | 7 | (7.4%) |
| Weight decreased | 1 | (0.6%) | 2 | (1.2%) | 5 | (2.8%) | 2 | (2.1%) |
| Metabolism and | 4 | (2.2%) | 1 | (0.6%) | 5 | (2.8%) | 1 | (1.1%) |
| nutrition disorders | | | | | | | | |
| Musculoskeletal and | 2 | (1.1%) | 4 | (2.3%) | 2 | (1.1%) | 2 | (2.1%) |
| connective tissue | | | | | | | | |
| disorders | | | | | | | | |
| Nervous system: | | | | | | | | |
| Any | 3 | (1.7%) | 3 | (1.7%) | 17 | (9.6%) | 4 | (4.3%) |
| Headache | 2 | (1.1%) | 1 | (0.6%) | 15 | (8.5%) | 3 | (3.2%) |
| Psychiatric disorders | 3 | (1.7%) | 0 | (0.0%) | 2 | (1.1%) | 2 | (2.1%) |
| Renal and urinary | 1 | (0.6%) | 2 | (1.2%) | 1 | (0.6%) | 2 | (2.1%) |
| disorders | | | | | | | | |
| Skin/sc tissue disorder: | | | | | | | | |
| Any | 5 | (2.8%) | 7 | (4.0%) | 6 | (3.4%) | 7 | (7.4%) |
| Pruritus | 1 | (0.6%) | 2 | (1.2%) | 4 | (2.3%) | 2 | (2.1%) |

GI = gastrointestinal,
sc = subcutaneous
^{a} Includes instances where the incidence in the MedRA SOC (system organ class) was >2%.
^{b}Includes 151 patients who received piclidenoson 2 mg BID in Segment 1 of the study plus 28 patients who received placebo in Segment 1 and were re-randomized to piclidenoson 2 mg BID at Week 16.
^{c}Includes 142 patients who received piclidenoson 3 mg BID in Segment 1 of the study plus 31 patients who received placebo in Segment 1 and were re-randomized to piclidenoson 3 mg BID at Week 16.
^{d}Includes 142 patients who received apremilast in Segment 1 of the study plus 35 patients who received placebo in Segment 1 and were re-randomized to apremilast at Week 16.
^{e}Patients received placebo for 16 weeks only before re-randomization to an active treatment.

### Pharmacokinetics (PK)

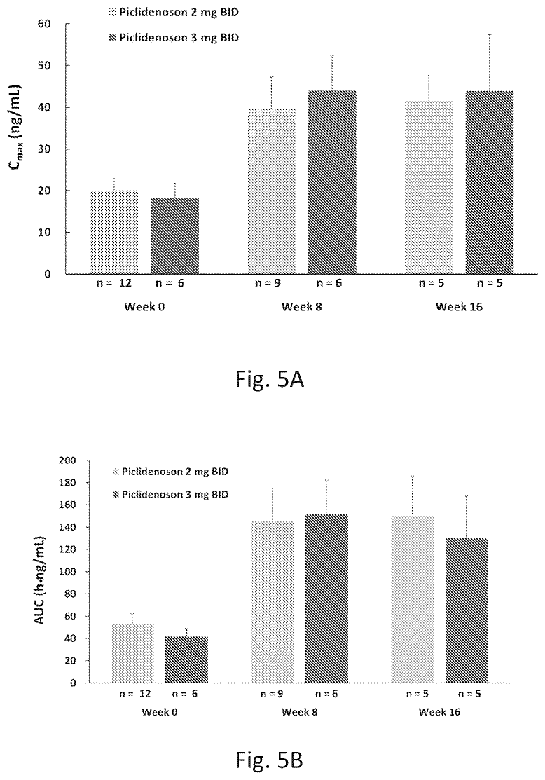

PK data demonstrated that the exposure to piclidenoson as measured by both Cₘₐₓ and AUC (area under curve) was similar in the 2 mg BID and 3 mg BID arms (FIG. 5).

The invention claimed is:

1-15. (canceled)

16. A method for treatment of psoriasis comprising administering 1-[N⁶-(3-iodobenzyl)-adenin-9-yl]-β-D-ribofuronamide (IB-MECA) or a pharmaceutical composition comprising IB-MECA to a patient in need thereof in a daily dose of about 6 milligrams (mg).

17. The method of claim 16 wherein said IB-MECA or said pharmaceutical composition are administered orally.

18. The method of claim 16, wherein said IB-MECA or said pharmaceutical composition are administered once or twice a day, the total daily dose being about 6 mg.

19. The method of claim 16, wherein said IB-MECA or said pharmaceutical composition are administered as a solid dosage form comprising 3 mg IB-MECA which is administered twice a day.

20. The method of claim 19 wherein said solid dosage form is selected from pills, tablets, and capsules.

21. The method of claim 16 wherein said psoriasis is moderate to severe plaque psoriasis.

22. A package comprising a pharmaceutical composition comprising 1-[N⁶-(3-iodobenzyl)-adenin-9-yl]-β-D-ribofuronamide (IB-MECA) and instructions for administration of the pharmaceutical composition to a subject in need of treatment of psoriasis at a daily dose of IB-MECA of about 6 mg.

23. The package of claim 22, wherein the pharmaceutical composition comprises 3 mg IB-MECA and said instructions comprises administration of the pharmaceutical composition to the subject twice a day.

24. The package of claim 22 wherein said psoriasis is moderate to severe plaque psoriasis.
